(19) United States
(12) Patent Application Publication
(10) Pub. No.: US 2005/0055166 A1
(43) Pub. Date: Mar. 10, 2005

(54) Nonlinear modeling of gene networks from time series gene expression data

(76) Inventors: Satoru Miyano, Tokyo (JP); Seiya Imoto, Tokyo (JP); Sun Yong Kim, Saitama Prefecture (JP)

Correspondence Address: Sheldon R. Meyer;FLIESLER DUBB MEYER & LOVEJOY LLP, Fourth Floor, Four Embarcadero Center, San Francisco, CA, 94111-4156, US

(21) Appl. No.: 10/716,330

(22) Filed: Nov. 18, 2003

Related U.S. Application Data
(60) Provisional application No. 60/427,448, filed on Nov. 19, 2002.

(51) Int. Cl. G06F 19/00; G01N 33/48; G01N 33/50

(52) U.S. Cl. 702/20

(57) ABSTRACT

Embodiments of this invention include application of new inferential methods to analysis of complex biological information, including gene networks. In some embodiments, time course data obtained simultaneously for a number of genes in an organism. New methods include modifications of Bayesian inferential methods and application of those methods to determining cause and effect relationships between expressed genes, and in some embodiments, for determining upstream effectors of regulated genes. Additional modifications of Bayesian methods include use of time course data to infer causal relationships between expressed genes. Other embodiments include the use of bootstrapping methods and determination of edge effects to more accurately provide network information between expressed genes. Information about gene networks can be stored in a memory device and can be transmitted to an output device, or can be transmitted to remote location.

### RELATED APPLICATION

This application claims priority under 35 U.S.C. §119(e) to U.S. Provisional Application Ser. No. 60/427,448 filed Nov. 19, 2002. This application is herein incorporated fully by reference.

### FIELD OF THE INVENTION

This invention relates to the use of Bayesian models with nonparametric regression to infer network relationships between genes from time series studies of gene expression. In particular, the invention relates to methods involving minimizing a criterion, BNRC_{dynamic} to infer optimal network relationships.

### BACKGROUND

One of the most important aspects of current research and development in the life sciences, medicine, drug discovery and development and pharmaceutical industries is the need to develop methods and devices for interpreting large amounts of raw data and drawing conclusions based on such data. Bioinformatics has contributed substantially to the understanding of systems biology and promises to produce even greater understanding of the complex relationships between components of living systems. In particular, with the advent of new methods for rapidly detecting expressed genes and for quantifying expression of genes, bioinformatics can be used to predict potential therapeutic targets even without knowing with certainty, the exact roles a particular gene(s) may play in the biology of an organism.

Simulation of genetic systems is a central topic of systems biology. Because simulations can be based on biological knowledge, a network estimation method can support biological simulation by predicting or inferring previously unknown relationships.

In particular, development of microarray technology has permitted studies of expression of a large number of genes from a variety of organisms. A very large amount of raw data can be obtained from a number of genes from an organism, and gene expression can be studied by intervention by either mutation, disease or drugs. Finding that a particular gene's expression is increased in a particular disease or in response to a particular intervention may lead one to believe that that gene is directly involved in the disease process or drug response. However, in biological organisms genes rarely are independently regulated by any such intervention, in that many genes can be affected by a particular intervention. Because a large number of different genes may be so affected, understanding the cause and effect relationships between genes in such studies is very difficult. Thus, much effort is being expended to develop methods for determining cause and effect relationships between genes, which genes are central to a biological phenomenon, and which genes' expression(s) are peripheral to the biological process under study. Although such peripheral gene's expression maybe useful as a marker of a biological or pathophysiological condition, if such a gene is not central to physiological or pathophysiological conditions, developing drugs based on such genes may not be worth the efforts. In contrast, for genes identified to be central to a process, development of drugs or other interventions may be crucial to developing treatments for conditions associated with altered expression of genes.

Development of bayesian network analysis for estimating a gene network from microarray gene expression data has received considerable attention and many successful investigations have been reported (Friedman et al [13]; Imoto etal [14]; Pe'er et al. [18] and our own work [U.S. patent application Ser. No. 10/259,723 herein incorporated fully by reference].

However, a shortcoming of traditional Bayesian network models is that they cannot construct cyclic networks, while certain real gene regulatory mechanisms have cyclic components. Recently, a dynamic Bayesian network model (Bilmes et al. [3]; Friedman et al [12]; Someren et al [19] has been propsed for constructing a gene network with cyclic regulatory components. Dynamic Bayesian network is based on time series data, and usually the data can be discritized into several classes. Thus, a dynamic network model can depend on the setting of the thresholds for the discritizing process, and unfortunately, the discritization can lead to loss of information. Imoto et al. [14, 15] proposed a network estimation method based on a Bayesian network and nonparametric regression for a solution to avoid discretization and for capturing non-linear relations among genes. However, Bayesian networks and nonparametric regression models [14, 15] still may not adequately solve networks having cyclic regulatory components.

### SUMMARY

In certain embodiments, this invention includes the use of time-series expression data in a Bayesian network model with nonparametric regression. Using time series expression data, we can identify cyclic regulatory components. In other embodiments, time delay information can be incorporated into a Bayesian/nonparametric regression model, which then can extract even nonlinear relations among genes. In certain of these embodiments, an ordinal differential equation model can be used as an alternative. We also have developed new criteria for choosing an optimal network from a Bayesian statistical point ofview. Such criteria can optimize a network structure based on data having noise.

### BRIEF DESCRIPTION OF THE FIGURES

This invention is described with reference to specific embodiments thereof. Additional aspects of the invention are found the Examples and in the Figures, in which:

FIG. 1 depicts a schematic illustration of time dynamics in gene expression.

FIGS. 2a and 2b depict diagrams of network relationships of genes involved in cell cycle regulation in yeast, compiled in KEGG.

FIG. 2a depicts genes in cyclin-dependent protein kinase pathways.

FIG. 2b depicts network relationships between genes described in FIG. 2a involved in regulating cyclin-dependent protein kinases.

FIGS. 3a-3c depict diagrams of network relationships of yeast genes involved in metabolic pathways.

FIG. 3a depicts several genes involved in metabolic pathways.

FIG. 3b depicts network relationships between genes described in FIG. 3a derived from a Bayesian/nonparametric regression model.

FIG. 3c depicts network relationships between genes described in FIG. 3a derived from a dynamic Bayesian/nonparametric regression model.

### DETAILED DESCRIPTION

In general, a dynamic Bayesian network model can be obtained using any suitable method for determining gene expression. In certain embodiments, microarray experiments are desirable because a large number of genes can be studied from a single sample applied to the array, making relative differences in gene expression easy to determine. It maybe desirable to improve accuracy of microarray methods by subtracting background signals from the signal reflecting true gene expression and/or correcting for inherent differences in labels used to measure gene expression (e.g., cy3/cy5)

Using a Bayesian network framework, we consider a gene as a random variable and decompose the joint probability into the product of conditional probabilities. For example, if we have a series of observations of the random vector, we can denote the probability of obtaining a given observation can depend upon the conditional probability densities. In certain embodiments, one can use nonparametric regression models for capturing the relationships between the variables. A variety of graphic tools can be used to elucidate the relationships. For example, polynomials, Fourier series, regression spline gases, B-spline bases, wavelet bases and the like can be used for defining a graph of gene relationships. Certain methods to elucidate network relationships are disclosed in U.S. patent application Ser. No. 10/259,723, herein incorporated fully by reference. One difficulty in selecting a proper graph is to properly evaluate variance and noise in the system.

In some embodiments of this invention, networks can be constructed using Bayesian estimation with nonparametric regression using data from time series studies. In many gene networks, an intervention leads to alteration in expression of certain genes before alterations in other genes are observed. One may infer that expression of certain genes after an intervention that occur later in time, may be causally related to genes whose expression is early. Time series information is useful to define “early” or genes and “late” or gene. It is unlikely that an alteration in expression of a late gene could be a cause of an alteration of expression of an early gene, whose expression is altered sooner in time than that of a late gene. Although this presumption may not apply in all cases, it is more probable that early genes are more likely “upstream” in a network than are late genes, which are more likely to be “downstream” genes. Therefore, time relations of gene expression can be useful to modify Bayesian estimation and nonparametric regression to provide a more reliable network solution.

In aspects of this invention, we extend the Bayesian network and nonparametric regression model to a dynamic Bayesian network model, which can be used to construct cyclic relationships when one has time series gene expression data. Information on time delay between changes in gene expression can be included in a model easily, and the model can extract even nonlinear relations among genes easily.

In certain embodiments, for constructing a gene network with cyclic regulatory components, an ordinal differential equation model (Chen et al. [5]; de Hoon et al. [8] can be used. However, this model is based on a linear system and maybe unsuitable for capturing complex phenomena. We have derived a new criterion for choosing an optimal network from Bayesian statistical point of view [2]. The criterion can optimize network structure, which gives the best representation of the gene interactions described by the data with noise. The new criterion is herein termed BNRC_{dynamic}.

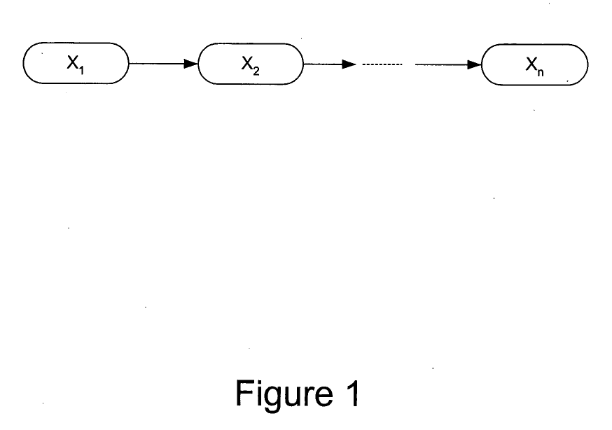

BNRC_{dynamic} can be evaluated using a first-order Markov relation as illustrated in FIG. 1. In such a relationship, an upstream gene X₁ is depicted as having an effect (right arrow) on one or more downstream genes X₂, which has an effect on X₃ (not shown), and so on, until an effect on Xₙ is observed. In situations in which X₁ has no “upstream” gene of its own, X₁ is termed a “parent” gene within the network. Genes under influence of a parent gene are termed “target” genes. Note that the use of “target gene” in this context is not to be confused with a gene that is a target for intervention, such as by a potential drug. In fact, parent genes may be targets for therapeutic intervention. Under this scheme, an effect on Xₙ cannot be observed until effects on X₁, X₂, etc. have been elicited. Note that FIG. 1 illustrates a “series” cause/effect relationship, without parallel or feedback systems are present, whereas in many genetic systems, there are series effects, and “parallel” effects, in which two or more genes can either be affected by an upstream gene, and/or can themselves affect a downstream gene. Moreover, circular effects (“feedback”) can be present, in which a gene Xₐ can affect another gene X_{b}, which can affect X_{c}, which itself can affect Xₐ (or X_{b}). Moreover, such feedback maybe either positive, in which X_{c} stimulates Xₐ or “negative” in which X_{c} inhibits Xₐ. Further complexities can arise in situations in which both series, parallel, positive feedback and negative feedback relationships are present.

In general, relationships between time points may be arbitrary, but in some cases it can be advantageous to use pre-selected time points based on knowledge of the biological effects of the genes and their expression dynamics under study. Under first order conditions, a joint probability can be decomposed as shown in equation (1) in Example 1 below. A conditional probability can then be decomposed into the product of conditional probabilities using equation (2) in Example 1. Equations (1) and (2) can hold and the density function can be used instead of a probability measure. Therefore, the dynamic Bayesian network can be represented, for example, using densities described in Example 1 to arrive at the local network structure of a gene and its parent genes according to equation (3) in Example 1.

A dynamic Bayesian model with nonparametric regression can be applied, for example, as described in Example 2. Once experimental data is collected, a the solution to the network can be considered to be a statistical model selection problem. In certain embodiments, we can solve this problem using Bayesian approach and derive a criterion for evaluating the goodness of the dynamic Bayesian network and nonparametric regression methods. Assuming a prior distribution, marginal likelihood and posterior probability can be determined according to equation (4) in Example 2. Subsequent construction of a genetic network involves computation of a high dimensional integral as depicted in equation (4). In some embodiments, a Laplace method for integrals, for example, can be used to approximate the integral. Therefore, the criterion BNRC_{dynamic} as shown in equation (5) in Example 2 can be solved.

To apply BNRC_{dynamic} to an experimental system, cDNA microarray data, for example, can be obtained experimentally at a number of time points after affecting the genetic system. To smooth curves, we can use spline functions, for example B-splines as depicted in Example 3. BNRC_{dynamic} can be decomposed according to equation (6) in Example 3. Optimal network relationships are obtained when BNRC_{dynamic} is minimized.

Using dynamic Bayesian network models with nonparametric regression and the criterion BNRC_{dynamic}, we can formulate a network learning process. However, determining which genes are parent genes and which are target genes can be time consuming when all possible gene combinations and relationships are considered. To reduce the number of analyses needed, we can select candidate parent genes. Subsequently a greedy hill-climbing algorithm can be used. BNRC_{dynamic} is calculated and then an addition parent gene is either added or deleted, and BNRC_{dynamic} is re-evaluated according to Step 2 in Example 3. The process is repeated until an appropriate convergence is found. Then, the order of computation is permutated and BNRC_{dynamic} is reevaluated. The optimal network give the smallest BNRC_{dynamic}.

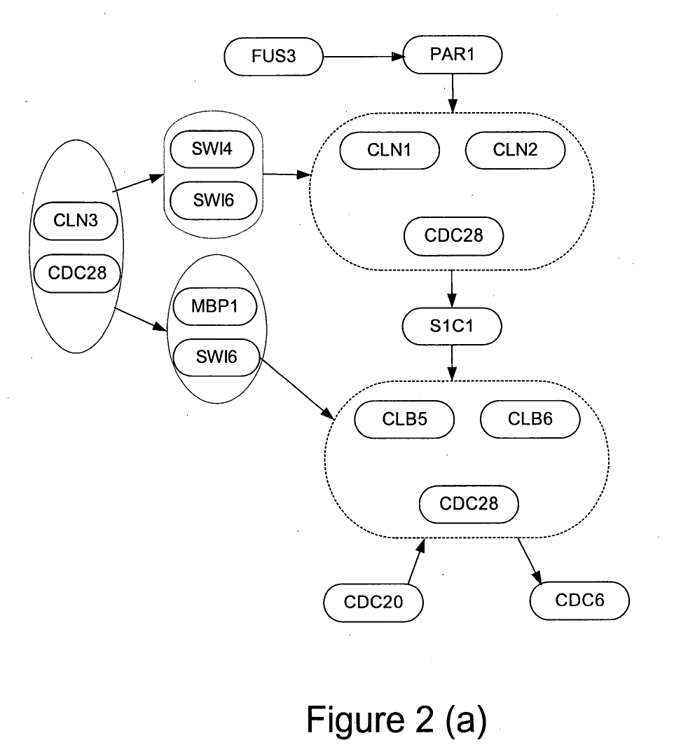

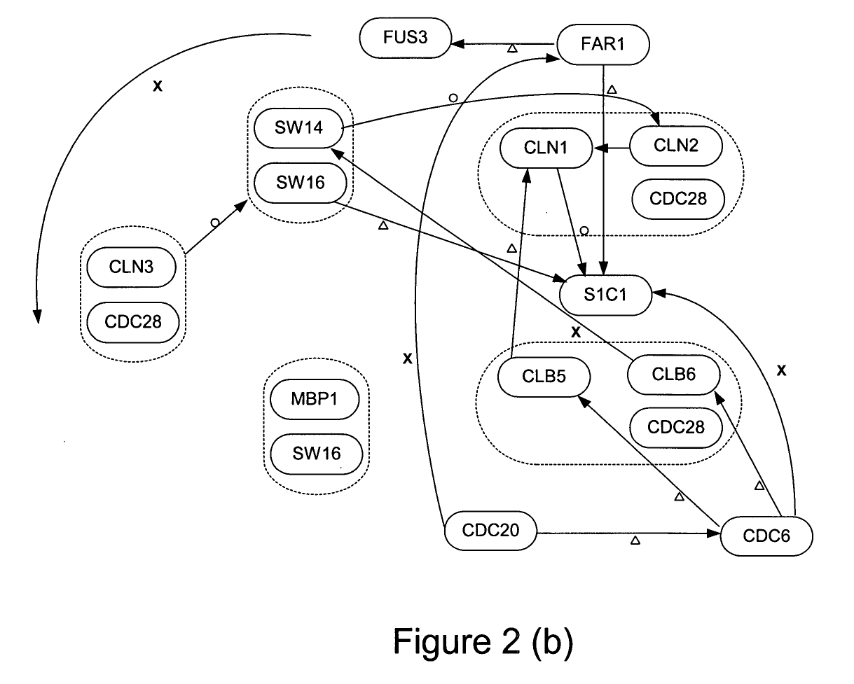

A specific illustration of the above methods are shown in Example 4 in FIGS. 2a and 2b. The efficiencies of the methods are shown through analysis of gene expression data from Saccharomyces cerevisiae. FIG. 2a depicts a group of S. cerevisiae genes involved in regulation of cell cycle. The genes are depicted as grouped based in the overall metabolic pathways involved and focus on the cyclin-dependent protein kinase gene (YBR160w). Note that the parent/target gene network relationships are unknown based on FIG. 2a. In contrast, using methods of this invention, network relationships of those genes can be evaluated and are depicted in FIG. 2b.

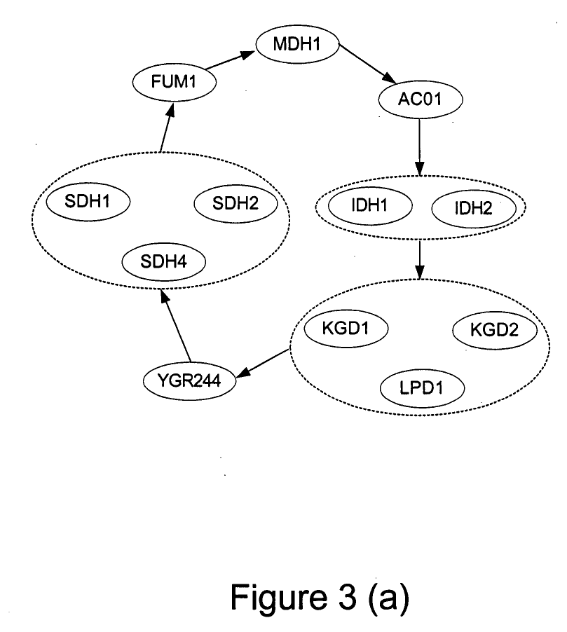

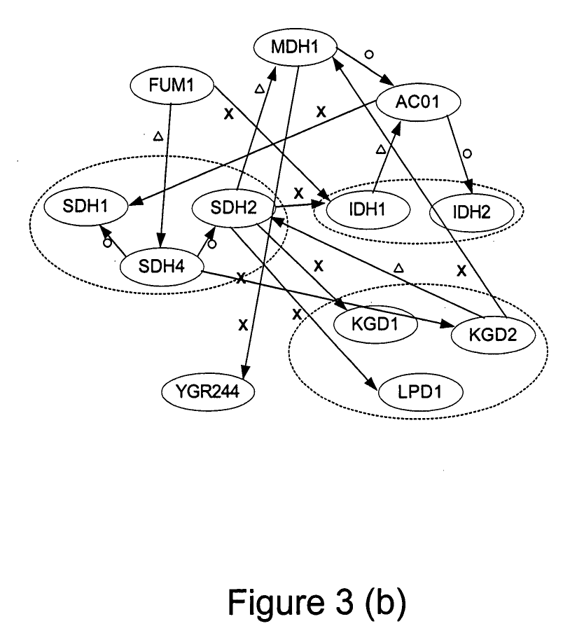

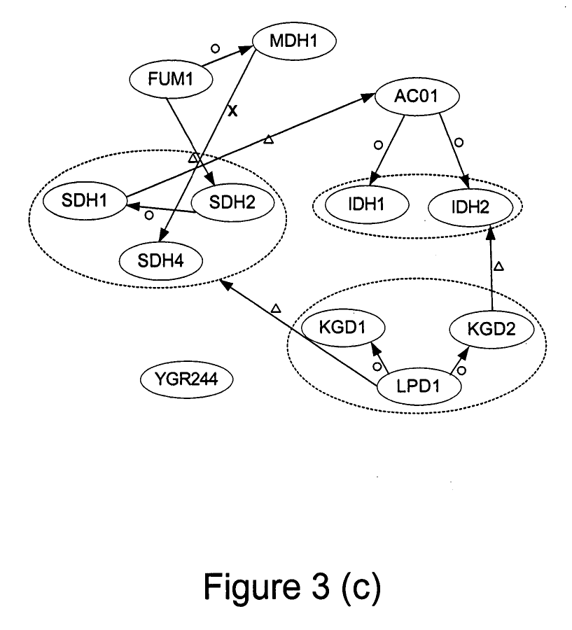

Another example is depicted in FIGS. 3a-3c. FIG. 3a depicts genes involved in metabolic pathways. FIG. 3a shows no gene network relationships. FIG. 3b depicts a network solution obtained using Bayesian network analysis with nonparametric regression, but without consideration of BNRC_{dynamic}. FIG. 3c depicts a network solution obtained by minimizing BNRC_{dynamic}. Note that in FIG. 3b, the network relationships are simpler, and compared to those depicted in FIG. 3b, there are many fewer false positive relationships (“x”).

Boundaries between groups of genes in a network can be determined using methods known in the art, for example, bootstrap methods. Such methods include determining the intensity of an edge

using the following steps.

(1) providing a bootstrap gene expression matrix by randomly sampling a number of times, with replacement, from the original gene library expression data;
(2) estimating the genetic network for genes and geneⱼ;
(3) repeating steps (1) and (2) T times, thereby producing T genetic networks; and
(4) calculating the bootstrap edge intensity between geneᵢ and geneⱼ as (t₁+t₂)/T.

Advantages of the new methods compared with other network estimation methods such as Bayesian and Boolean Networks include: (1) time information can be incorporated easily; (2) microarray data can be analyzed as continuous data without extra data pre-treatments such as discretization; and (3) fewer false positive relationships are found. Even nonlinear relations can be detected and modeled by embodiments of this invention. Methods of this invention are useful for analyzing genetic networks and for development of new pharmaceuticals which target particularly genes that control genetic expression of important genes. Thus, methods of this invention can decrease the time needed to identify drug targets and therefore can decrease the time needed to develop new treatments.

Other aspects of methods of this invention are described in the Examples below.

### EXAMPLES

The examples presented below represent specific embodiments of this invention. Other aspects of the invention can be developed by persons of ordinary skill in the art without undue experimentation. All such embodiments are considered part of this invention.

### Example 1

### Bayesian Network and Nonparametric Regression

Suppose that we have an n×p microarray gene expression data matrix X, where n and p are the numbers of microarrays and genes, respectively. Usually, the number of genes p is much larger than the number of microarrays, n. In the estimation of a gene network based on the Bayesian network, a gene is considered as a random variable. When we model a gene network by using statistical models described by the density or probability function, the statistical model should include p random variables. However, we have only n samples and n is usually much smaller than p. In such case, the inference of the model is quite difficult or impossible, because the model has many parameters and the number of samples is not enough for estimating the parameters. The Bayesian network model has been advocated in such modeling.

In the context of the dynamic Bayesian network, we consider the time series data and the ith column vector xᵢ of X corresponds to the states of p genes at time i. As for the time dependency, we consider the first order Markov relation described in FIG. 1. Under this condition, the joint probability can be decomposed as follows:
P(X₁₁, . . . , Xₙₚ)=P(X₁)P(X₂|X₁)× . . . ×P(X₁Xₙ₋₁), (1)
where Xᵢ=(Xᵢ₁, . . . , Xᵢₚ) is a random variable vector of p genes at time i. The conditional probability P(Xᵢ|Xᵢ₋₁) can also be decomposed into the product of conditional probabilities of the form
P(Xᵢ|Xᵢ₋₁)=P(Xᵢ₁|P_{i-1,1})× . . . ×P(Xᵢₚ|P_{i-1,p}), (2)
where P_{i-1,j} is the state vector of the parent genes of jth gene at time i−1. The equations (1) and (2) hold when we use the density function instead of the probability measure Hence, the dynamic Bayesian network can then be represented by using densities as follows:

$$\begin{matrix}f ( {x}_{11} , … , {x}_{np} ) = {f}_{1} ( {x}_{1} ) {f}_{2} ( {x}_{2} \vert {x}_{1} ) \times ⋯ \times {f}_{n} ( {x}_{n} \vert {x}_{n - 1} ) \\ = {f}_{1} ( {x}_{1} ) {\prod }_{i = 2}^{n} {g}_{1} ( {x}_{i1} \vert {p}_{i - 1 , 1} ) \times ⋯ \times {g}_{p} ( {x}_{ip} \vert {p}_{i - 1 , p} ) \\ = {f}_{1} ( {x}_{1} ) {\prod }_{j = 1}^{p} \{ {\prod }_{i = 2}^{n} {g}_{j} ( {x}_{ij} \vert {p}_{i - 1 , j} ) \} .\end{matrix}$$

Here we have the decomposition from (2)
fᵢ(xᵢ|xᵢ₋₁)=g₁(xᵢₗ|p_{i-1,1})× . . . ×gₚ(xᵢₚ|p_{i-1,p}),
where p_{i-1,j}=(p_{i-1,1}^{(j)}, . . . , p_{i-1,qj}^{(j)}) is a qⱼ-dimensional observation vector of parent genes.

For modeling the relationship between xᵢⱼ and p_{i-1,j}, we use the nonparametric additive regression model as follows:
xᵢⱼ=mⱼ₁(p_{i=1,1}^{(j)})+ . . . +m_{jqj}(p_{i=1,qj}^{(j)})+εᵢⱼ,
where εᵢⱼ depends independently and normally on mean 0 and variance σⱼ². Here, mⱼₖ(•) is a smooth function from R to R and can be expresse(d by using the linear combination of basis functions

$${m}_{jk} ( {p}_{i - 1 , k}^{( j )} ) = {\sum }_{m = 1}^{{M}_{jk}} {γ}_{mk}^{( j )} {b}_{mk}^{( j )} ( {p}_{i - 1 , k}^{( j )} ) , k = 1 , … , {q}_{j} ,$$

where γ₁ₖ^{(j)}, . . . , γ_{M}_{ⱼₖ}ₖ^{(j)} are unknown coefficient parameters and {b₁ₖ^{(j)}(•), . . . , b_{M}_{ⱼₖ}ₖ(•)} is the prescribed set of basis functions. Then we define a dynamic Bayesian network and nonparametric regression model of the form

$$\begin{matrix}f ( {x}_{11} , … , {x}_{np} ; {θ}_{G} ) = {f}_{1} ( {x}_{1} ) {\prod }_{j = 1}^{p} \\ [ {\prod }_{i = 2}^{n} \frac{1}{\sqrt{2 π {σ}_{j}^{2}}} exp \{ - \frac{{( {x}_{ij} - μ ( {p}_{i - 1 , j} ) )}^{2}}{2 {σ}_{j}^{2}} \} ] ,\end{matrix}$$

where μ(p_{i-1,j})=mⱼ₁(p_{i-1,1}^{(j)})+ . . . +m_{jq}_{ⱼ}(p_{i-1,jq}_{ⱼ}^{(j)}). When jth gene has no parent genes, μ(p_{i-1,j}) is resulted in the constant μⱼ.

We assume f₁(x₁)=g₁(x₁₁)× . . . ×g₁(x₁ₚ) and the joint density f(x₁₁, . . . , xₙₚᵢ; θ_{G}) can then be rewritten as

$$\begin{matrix}f ( {x}_{11} , … , {x}_{np} ; {θ}_{G} ) = {\prod }_{j = 1}^{p} [ {g}_{1} ( {x}_{1 j} ) {\prod }_{i = 2}^{n} \frac{1}{\sqrt{2 π {σ}_{j}^{2}}} exp \\ \{ - \frac{{( {x}_{ij} - μ ( {p}_{i - 1 , j} ) )}^{2}}{2 {σ}_{j}^{2}} \} ] \\ = {\prod }_{j = 1}^{p} {\prod }_{i = 1}^{n} {g}_{j} ( {x}_{ij} \vert {p}_{i - 1 , j} ; {θ}_{j} ) ,\end{matrix} \qquad ( 3 )$$

where p₀ⱼ=θ. Thus, gⱼ(xᵢⱼ|p_{i-1,j}; θⱼ) represents the local structure of jth gene and its parent genes.

### Example 2

### Derivation of a Criterion for Selecting a Network

The dynamic Bayesian network and nonparametric regression model introduced in the previous section can be constructed when we fic the network structure and estimated by a suitable procedure. However, the gene network is generally unknown and we should estimate an optimal network based on the data. This problem can be viewed as a statistical model selection problem (see e.g., Akaike [1]; Konishi and Kitagawa [17]; Burnham and Anderson [4]; Konishi [16]). We solve this problem from the Bayesian statistical approach and derive a criterion for evaluating the goodness of the dynamic Bayesian network and nonparametric regression model.

Let π(θ_{G}|λ) be a prior distribution on the parameter θ_{G} in the dynamic Bayesian network and nonparametric regression model and let log π(θ_{G}|λ)=O(n). The marginal likelihood can be represented as
∫f(x₁₁, . . . , xₙₚ; θ_{G})π(θ_{G}|λ)dθ_{G}.
Thus, when the data is given, the posterior probability of the network G is

$${π}_{post} ( G \vert X ) = \frac{\begin{matrix}{π}_{prior} ( G ) \\ \int f ( {x}_{11} , … , {x}_{np} ; {θ}_{G} ) π ( {θ}_{G} \vert λ ) ⅆ {θ}_{G}\end{matrix}}{\begin{matrix}{Σ}_{G} \\ \{ {π}_{prior} ( G ) \int f ( {x}_{11} , … , {x}_{np} ; {θ}_{G} ) π ( {θ}_{G} \vert λ ) ⅆ {θ}_{G} \}\end{matrix}} , \qquad ( 4 )$$

where πprior(G) is the prior probability of the network G. The denominator of (4) does not relate to model evaluation. Therefore, the evaluation of the network depends on the magnitude of numerator. Hence, we can choose an optimal network as the maximizer of
πₚᵣᵢₒᵣ(G)∫f(x₁₁, . . . , xₙₚᵢ; θ_{G})π(θ_{G}|λ)dθ_{G}.
It is clear that the essential point for constructing a network selection criterion is how to compute the high dimensional integral. Imoto et al. [14, 15] used the Laplace approximation for integrals (see also Tinerey and Kadane [21]; Davison [6]) and we can apply this technique to the dynamic Bayesian network model and nonparametric regression directly. Hence, we have a criterion, named BNRC_{dynamic} of the form

$$\begin{matrix}{BNRC}_{dynamic} ( G ) = - 2 log \{ {π}_{prior} ( G ) \int f ( {x}_{11} , … , {x}_{np} ; {θ}_{G} ) π \\ ( {θ}_{G} \vert λ ) ⅆ {θ}_{G} \} \\ \approx - 2 log {π}_{prior} ( G ) - r log ( 2 π / n ) + \\ log \vert {J}_{λ} ( {\hat{θ}}_{G} ) \vert - 2 {nl}_{λ} ( {\hat{θ}}_{G} \vert {X}_{n} ) ,\end{matrix} \qquad ( 5 )$$

where r is the dimension of θ_{G},
l_{λ}(θ_{G}51 Xₙ)=log f(x₁₁, . . . , xₙₚᵢ; θ_{G})/n+log π(θ_{G}|λ)/n,
J_{λ}(θ_{G})=∂²{l_{λ}(θ_{G}|Xₙ)}/∂θ_{G}θ^{T}_{G}
and θ_{G} is the mode of l_{λ}(θ_{G}|Xₙ). The optimal graph is chosen such that the criterion BNRC_{dynamic} (5) is minimal.

### EXAMPLE 3

### Estimation of a Gene Network

In this section, we show a concrete strategy for estimating a gene network from cDNA microarray time series gene expression data.

3.1 Nonparametric Regression

We use the basis function approach for constructing the smooth function mⱼₖ(•) described in Section 2. In this paper we use B-splines (de Boor [7]) as the basis functions. De Boor's algorithm (de Boor [7], Chapter 10, p.130 (3)) is a useful method for computing B-splines of any degree. We use 20 B-splines with equidistance knots (see also, Dierckx [10]; Eiler and Marx [11] for the details of B-spline).

3.2 Prior Distribution on the Parameter in the Model

For the prior distribution on the parameter θ_{G}, suppose that the parameter vectors θⱼ are independent one another, the prior distribution can then be decomposed as π(θ_{G}|λ)=Πⱼ₌₁^{p}πⱼ(θⱼ|λⱼ). Suppose that the prior distribution πⱼ(θⱼ|λⱼ) is factorized as πⱼ(θⱼ|λⱼ)=Π_{l<1}^{qj}πⱼₖ(γⱼₖ|λⱼₖ), where λⱼₖ are hyper parameters. We use a singular Mⱼₖ variate normal distribution as the prior distribution on γⱼₖ,

$${π}_{jk} ( {γ}_{jk} \vert {λ}_{jk} ) = {( \frac{2 π}{n {λ}_{jk}} )}^{- ( {M}_{jk} - 2 ) / 2} {\vert {K}_{jk} \vert}_{+}^{1 / 2} exp ( - \frac{n {λ}_{jk}}{2} {γ}_{jk}^{T} {K}_{jk} {γ}_{jk} ) ,$$

where Kⱼₖ is an Mⱼₖ×Mⱼₖ symmetric positive semidefinite matrix satisfying

$${γ}_{jk}^{T} {K}_{jk} {γ}_{jk} = {\sum }_{α = 3}^{{M}_{jk}} {( {γ}_{α k}^{( j )} - 2 {γ}_{α - 1 , k}^{( j )} + {γ}_{α - 2 , k}^{( j )} )}^{2} .$$

This setting of the prior distribution on θ_{G} is the same as Imoto et al. [14, 15] nd the details are in those papers.

3.3 Proposed Criterion

By using the prior distributions in Section 4.2, the BNRC_{dynamic} can be decomposed as follows:

$${BNRC}_{dynamic} = {\sum }_{j = 1}^{p} {BNRC}_{dynamic}^{( j )} , \qquad ( 6 )$$

where BNRC_{dynamic}^{(j)} is a local criterion score of jth gene and is defined by

$$\begin{matrix}{BNRC}_{dynamic}^{( j )} = - 2 log \{ \int {π}_{prior} ( {L}_{j} ) {\prod }_{i = 1}^{n} {g}_{j} ( {x}_{ij} \vert {p}_{i - 1 , j} ; {θ}_{j} ) {π}_{j} \\ ( {θ}_{j} \vert {λ}_{j} ) ⅆ {θ}_{j} \} \\ \approx - 2 log {π}_{prior} ( {L}_{j} ) - {r}_{j} log ( 2 π / n ) + log \vert {J}_{{λ}_{j}}^{( j )} ( {\hat{θ}}_{j} ) \vert - \\ 2 {nl}_{{λ}_{j}}^{( j )} ( {\hat{θ}}_{j} \vert X ) ,\end{matrix}$$

where rⱼ is the dimension of θⱼ,

$${l}_{{λ}_{j}}^{( j )} ( {\hat{θ}}_{j} \vert X ) = {\sum }_{i = 1}^{n} log {g}_{j} ( {x}_{ij} \vert {p}_{i - 1 , j} ; {θ}_{j} ) / n + log π ( {θ}_{j} \vert {λ}_{j} ) / n ,$$

J_{λ}_{ⱼ}^{(j)}(\hat{θ}ⱼ)=−∂²{l_{λ}_{ⱼ}^{(j)}(\hat{θ}ⱼ|X)}/∂θⱼ∂θⱼ^{T}
and \hat{θ}ⱼ is the mode of l_{λ}_{ⱼ}^{(j)}(θⱼ|X). Here πₚᵣᵢₒᵣ(Lⱼ) are prior probabilities satisfying

$${\sum }_{j = 1}^{p} log {π}_{prior} ( {L}_{j} ) = log {π}_{prior} ( G ) .$$

We set the prior probability of local structure πₚᵣᵢₒᵣ(Lⱼ) as πₚᵣᵢₒᵣ(Lⱼ)=exp{−(The number of parent genes of j th gene)}.

By using the dynamic Bayesian network and nonparametric regression model together with the proposed criterion, BNRCd1/namic, we can formulate the network learning process as follows: it is clear from (3) and (6) that the optimization of network structure is equivalent to the choices of the parent genes that regulate the target genes. However, it is a time-consuming task when we consider all possible gene combinations as the parent genes. Therefore, we cut down the learning space by selecting candidate parent genes. After this step, a greedy hill climbing algorithm is employed for finding better networks. Our algorithm can be expressed as follows:

Step 1: Preprocessing Stage

We make the p×p matrix whose (i, j)th element is the BNRC score of the graph “geneᵢ→geneⱼ” and we define the candidate set of parent genes of geneⱼ that gives small BNRC score. We set the number of elements of the candidate set of parent genes 10.

Step 2: Learning Stage

For a greedy hill-climbing algorithm, we start form the empty network and repeat the following steps:
Step2-1: For genes, implement one from two procedures that add a parent gene, delete a parent gene, which gives smaller BNRC_{dynamic} score.
Step2-2: Repeat Step2-1 for prescribed computational order of genes until suitable convergence criterion is satisfied.
Step2-9: Permute the computational order for finding better solution and repeat Step2-1 and 2-2.
Step2-4: We choose the optimal network that gives the smallest BNRC_{dyanmic} score.

### Example 4

### Computational Experiment

We demonstrated one embodiment of this invention through the analysis of the Saccharomyces cerevisiae cell cycle gene expression data collected by Spellman et al. [20]. This data contains two short time series (two time points; cln3, clb2) and four medium time series (18, 24, 17 and 14 time points; alpha, cdc15, cdc28 and elu). In the estimation of a gene network, we used four medium time series. For combining four time series, we ignored the first observation of the target gene and last one of parent genes for each time series when we fit the nonparametric regression model.

At first, we focused on the cell cycle pathway compiled in KEGG database [22]. The target network is around CDC28 (YBR160w; cyclin-dependent protein kinase). This network contains 45 genes and the pathway registered in KEGG is shown in FIG. 2(a) and the estimated network is in FIG. 2(b). The edges in the dotted circles can be considered the correct edges. We thus modeled some correct relations. We denoted the correct estimation by the circle next to edge. The triangle represents the reverse or skip of correct direction. The “x” symbols represent incorrect relationships.

A second example used to demonstrate our methods is the metabolic pathway reported by DeRisi et al. [9]. This network contains 57 genes and the target pathway is shown in FIG. 3(a).

We applied a Bayesian network and nonparametric regression model [14,15] to this data and the resulting network is depicted in FIG. 3(b). The network of FIG. 3(c) was obtained by the dynamic Bayesian network and nonparametric regression model. It is difficult to estimate the metabolic pathway from cDNA microarray data. However, our model detected correct relationships between the genes. Compared with the Bayesian network and nonparametric regression, the number of false positives of this method depicted in FIG. 3(c) was much smaller than those depicted in FIG. 3(b) by the “x” symbols.

All references cited herein are incorporated herein in their entirety.

### REFERENCES

1. Akaike, J.: Information theory and an extension of the maximum likelihood principle. In: Petrov, B. N., Csaki, F. (eds.):2nd International Symposium on Information Theory. Akademiai Kiado, Budapest pp: 267-281 (1973).
2. Berger, J. O.: Statistical Decision theory and Bayesian analysis. Springer-Verlag New York (1985).
3. Bilmes, J. A.: Dynamic Bayesian multinets. Proc. 16th Conference on Uncertainty in Artificial Intelligence. pp: 38-45 (2000).
4. Burnham, K. P., Anderson, D. R.: Model selection and inference, a practical information-theoretical approach. Springer-Verlag New York (1988).
5. Chen, Tl., He, H. L., Church, G. M.: Modeling gene expression with differential equations. Proc. Pacific Symposium on Biocomputing 4: 29-40 (1999).
6. Davison, A. C.: Approximate predictive likelihood. Biometrika 73: 323-332 (1986).
7. DeBoor, C.: A pracitial guide to splines. Springer-Verlag Berlin (1978).
8. De Hoon, M. J. L., Imoto, S., Kobayashi, K., Ogasawara, N H., Miyano, S.: Inferring gene regulatory networks from time-ordered gene expression data of Bacillus subtilis using differential equations. Proc. Pacific Symposium on Biocomputing 8: 2003, in press.
9. DeRisi, J., Lyer, V. R., Brown, P. O.: Exploring the metabolic and gene control of gene expression on a genonmic scale. Science 278: 680-686 (1997).
10. Dierckx, P.: Curve and surface fitting with splines. Oxford (1993).
11. Eiler, P. H. C., Marx, B.: Flexible smoothing with B-splines and penalites (with discussion). Statistical Science 11:89-121 (1996).
12. Friedman, N., Murphy, K., Russell, S.: Learning the structure of dynamic probabilistic networks. Proc. Conf. On Uncertainty in Artificial Ingtelligence pp: 139-147 (1998).
13. Firedman, N., Linial, M I, Nachman, I., Pe'er, D.: Using Bayesian network to analyze expression data. J. Comp. Biol. 7: 601-620 (2000).
14. Imoto, S., Goto, T., Miyano, S I.: Estimation of gnetic networks and functional structures between genes by using Bayesian network and noparametric regression. Proc. Pacific Symposium on Biocomputing 7: 175-186 (2002).
15. Imoto, S., Kim, S., Goto, T., Aburatani, S., Tashiro, K., Kuhara, S., Mjiyano, S.: Bayesian network and nonparametric heteroscedastic regression for nonlinear modeling of genetic network. Proc. IEEE Computer Society Bioinformatics Conference; pp: 219-227 (2002).
16. Konishi, S.: Statistical model evaluation and information criteria. In: Ghosh, S. (ed.). Multivariate Analysis, Design of Experiments and Survey Sampling. Marcel Dekker, New York, pp: 369-399 (1999).
17. Konishi, S., Kitagawa, G.: Generalized information criteria in model selection. Biometrika 83: 875-890 (1996).
18. Pe'er, D., Regev, A., Elidan, G., Friedman, N.: Inferring subnetworks from perturbed expression profiles. Bioinformatics 17: 215-224 (ISBM 2001).
   19. Someren, E. V., Wessels, L., Reinders, M.: Linear modeling of genetic networks from experimental data. Bioinformatics 18: 355-366 (ISBM 2002).
20. Spellman, P. T., Sherlock, G., Zhang, M. Q., Iyer, V. R., Anders, K., Eisen, M. B., Brown, P. O., Botstein, D., Futcher, B.: Comprehensive identification of cell cycle-regulated genes of the yeast Saccharomyces cervisiae by microarray hybridization. Molecular Biology of the Cell 9: 3273-3297 (1998).
21. Tinerey, L., Kadane, J. B.: Accurate approximations for posterior moments and marginal densities. J. Amer. Statist. Assoc. 81: 82-86 (1986).

1. A method for constructing a gene network, comprising the steps of:
(a) providing a quantitative time course data library for a set of genes of an organism, said library including expression results based on time course of expression of each gene in said set of genes, quantifying an average effect and a measure of variability of each time point on each other of said genes;
(b) creating a gene expression matrix from said library;
(c) generating network relationships between said genes; and
(d) determining if one or more groups of genes is expressed differently from other of said groups of genes.

2. The method of claim 1, further comprising the step of:
(e) providing a Bayesian computational model, wherein said Bayesian model comprises minimizing a BNRC_{dynamic} criterion.

3. The method of claim 2, wherein said step of minimizing a BNRC_{dynamic} criterion comprises using a non-linear curve fitting method selected from the group consisting of polynomial bases, Fourier series, wavelet bases, regression spline bases and B-splines.

4. The method of claim 1, wherein said data library is created using time course study to alter gene expression.

5. The method of claim 2, wherein said step of minimizing said BNRC_{dynamic} criterion further comprises selecting a Bayesian mode using a backfitting algorithm.

6. The method of claim 2, wherein said step of minimizing a BNRC_{dynamic} criterion further comprises using Akaike's information criterion.

7. The method of claim 2, wherein said step of minimizing a BNRC_{dynamic} criterion further comprising using maximum likelihood estimation.

8. The method of claim 1, wherein said genes are associated with a cell cycle.

9. The method of claim 2, wherein said measure of variability is variance.

10. The method of claim 3, wherein said non-linear curve fitting method is a non-parametric method.

11. The method of claim 10, wherein said non-parametric method for minimizing a BNRC_{dynamic} criterion includes using heterogeneous error variances.

12. The method of claim 11, wherein said step of minimizing a BNRC_{dynamic} criterion further comprises the steps of:
(1) making a score matrix whose (i, j)^{th} element is the BNRC^{j}_{dynamic} score of the graph geneᵢ→geneⱼ;
(2) implementing one or more of add, remove and reverse which provides the smallest BNRC_{dynamic}; and
(3) repeating step 2 until the BNRC_{dynamic} does not reduce further.

13. The method of claim 11, wherein said step of minimizing a BNRC_{dynamic} criterion further comprises the step of applying a hill-climbing algorithm to minimize BNRC^{(j)}_{dynamic}.

14. The method of claim 11, wherein an intensity of the edge is determined using a bootstrap method.

15. The method of claim 14, wherein said bootstrap method comprises the steps of:
(1) providing a bootstrap gene expression matrix by randomly sampling a number of times, with replacement, from the original gene library expression data;
(2) estimating the genetic network for geneᵢ and geneⱼ;
(3) repeating steps (1) and (2) T times, thereby producing T genetic networks; and
(4) calculating the bootstrap edge intensity between genes and genes as (t₁+t₂)/T.

16. A method for elucidating a gene network, comprising the steps of:
(a) providing a raw data library of time-course gene expression data for a plurality of genes of an organism;
(b) subtracting background signal intensities from said raw data library;
(c) calculating the relative change in gene expression for each of said plurality of genes;
(d) analyzing the statistical significance of said relative in gene expression using Student's t-test; and
(e) fitting said changes in gene expression to a linear spline function.

17. The method of claim 16, further comprising the step of removing from consideration, those genes whose expression levels are sufficiently low so as to be determined predominantly by noise.

18. The method of claim 1, wherein said step of grouping comprises grouping said genes into one or more equivalence sets.

19. A method for estimating a gene network relationship, comprising the steps of:
(1) Making a p x p matrix whose (i,j)th element is a BNRC score of the graph geneᵢ→geneⱼ;
(2) select a candidate set of parent geneᵢ of geneⱼ that gives a small BNRC score
(3) select a computational order of said parent genes;
(4) repeat the following steps;
(4.1) for genes either add a parent gene or delete a parent gene;
(4.2) recalculate BNRC_{dynamic} score;
(4.3) repeat steps 3.1 and 3.2 until a suitable convergence criterion is satisfied;
(5) permute the computational order of said parent genes in step (3);
(6) repeat step (4); and
(7) repeat steps (5) and (6) until BNRC_{dynamic} is minimized.

20. A method for constructing a gene network model of a system containing a network of genes from time course gene expression data, said method comprises using a Bayesian computational model, wherein said Bayesian computational model comprises minimizing a BNRC_{dynamic} criterion.

21. The method of claim 20, wherein minimizing the BNRC_{dynamic} criterion comprises using a non-linear curve fitting method selected from the group consisting of polynomial bases, Fourier series, wavelet bases, regression spline bases and B-splines.

22. The method of claim 20, wherein minimizing the BNRC_{dynamic} criterion comprises selecting a Bayesian mode using a backfitting algorithm.

23. The method of claim 20, wherein minimizing the BNRC_{dynamic} criterion comprises using Akaike's information criterion.

24. The method of claim 20, wherein minimizing the BNRC_{dynamic} criterion comprises using maximum likelihood estimation.

25. The method of claim 20, wherein minimizing the BNRC_{dynamic} criterion comprises using a non-linear curve fitting method, wherein the non-linear curve fitting method is a non-parametric method.

26. The method of claim 25, wherein the non-parametric method includes using heterogeneous error variances.

27. The method of claim 26, wherein minimizing the BNRC_{dynamic} criterion further comprises the steps of:
(1) making a score matrix whose (i, j)^{th} element is the BNRC_{dynamic} score of the graph geneᵢ→geneⱼ;
(2) implementing one or more of add, remove and reverse which provides the smallest BNRC_{dynamic}; and
(3) repeating step 2 until the BNRC_{dynamic} does not reduce further.

28. The method of claim 26, wherein minimizing the BNRC_{dynamic} criterion further comprises the step of applying a hill-climbing algorithm to minimize BNRC^{(j)}_{dynamic}.

29. The method of claim 26, wherein an intensity of the edge is determined using a bootstrap method.

30. The method of claim 29, wherein said bootstrap method comprises the steps of:
(1) providing a bootstrap gene expression matrix by randomly sampling a number of times, with replacement, from the original gene library expression data;
(2) estimating the genetic network for geneᵢ and geneⱼ;
(3) repeating steps (1) and (2) T times, thereby producing T genetic networks; and
(4) calculating the bootstrap edge intensity between geneᵢ and geneⱼ as (t₁+t₂)/T.

31. A data file comprising a gene network model constructed by the method of claim 20.

32. The data file of claim 31 in a computer readable form.

33. The data file of claim 31 accessible from a remote location.

34. The data file of claim 31 accessible from an internet web location.

35. A method for identifying a target gene in a system containing a gene network, comprising:
(a) constructing a first and second gene network model using a Bayesian computational model,
wherein said Bayesian computational model comprises minimizing a BNRC_{dynamic} criterion,
wherein the first gene network model is obtained by analyzing a first gene expression profile and the second gene network model is obtained by analyzing a second gene expression profile, and
wherein the first gene expression profile is obtained from the system at a first time point and the second gene expression profile is obtained from the system at a second time point after said first time point, and
(b) analyzing the first and second gene network model using said Bayesian computational model, wherein the time course of gene expression is quantified, and
wherein a parent gene is identified as the target gene.

36. The method of claim 35, wherein the target gene is a parent gene.

37. The method of claim 35, wherein the target gene is a gene downstream from a parent gene.

38. A data file containing the identity of one or more target genes obtained according to the method of claim 35.

39. The data file of claim 38 in a computer readable form.

40. The data file of claim 38 accessible from a remote location.

41. The data file of claim 38 accessible from an internet web location.

42. A method of providing a service comprising
(1) receiving a data set from a party, said data set comprising time course expression data of a group of genes; and
(2) determining network relationships between genes in said group by minimizing a BNRC_{dynamic} criterion.

43. The method of claim 42, wherein receiving said data set comprises receiving the identity of at least one of said genes.

44. A method of providing a service comprising receiving an agent from a party, and identifying a target gene for the party using the gene network model constructed according to the method of claim 35.
